(19) United States
(12) Patent Application Publication
(10) Pub. No.: US 2016/0244771 A1
(43) Pub. Date: Aug. 25, 2016

(54) Method for inhibiting production of furanocoumarins in plants

(71) Applicant: UNIVERSITE DE LORRAINE, Nancy (FR)

(72) Inventors: Frederic BOURGAUD, VANDOEUVRE (FR); Alain HEHN, NEUVES MAISONS (FR); Alexandre OLRY, TONNOY (FR); Ryosuke MUNAKATA, KYOTO (JP); Kazufumi YAZAKI, KYOTO (JP); Mazaharu MIZUTANI, KYOTO (JP)

(21) Appl. No.: 15/049,360

(22) Filed: Feb. 22, 2016

Related U.S. Application Data
(60) Provisional application No. 62/120,492, filed on Feb. 25, 2015.

(51) Int. Cl. C12N 15/82; C12N 9/10; A01H 5/08

(52) U.S. Cl. CPC C12N 15/8218; A01H 5/0806; C12N 9/1085; C12Y 205/01

(57) ABSTRACT

A method for inhibiting production of furanocoumarins in a plant, includes inhibiting in the plant the expression of a protein named GfPT, the GfPT protein having coumarin-specific prenyltransferase activity and having at least 70% sequence identity with the polypeptide set forth in SEQ ID NO: 1.

### FIELD OF THE INVENTION

The present invention relates to method for inhibiting production of furanocoumarins in plants, in particular in plants belonging to the Rutaceae group.

### BACKGROUND OF THE INVENTION

Furanocoumarins constitute a class of phenolic secondary metabolites that are a sub-group of more than 1500 coumarin compounds. Furanocoumarins are essentially found in four higher-plant families (Apiaceae, Rutaceae, Fabaceae and Moraceae).

The metabolic profile of furanocoumarin differs largely among plant species, depending upon the age of the plant and the tissues concerned. There are two types of furanocoumarins derived from two parallel biosynthetic pathways: linear (i.e. psoralen derivatives) and angular furanocoumarins (i.e. angelicin derivatives. Many enzymes are involved in the furanocoumarin biosynthesis but only a few of them were already described at the molecular level such as two P450 enzymes (psoralen synthase and angelicin synthase) and a bergaptol O-methyltransferase. Recently a Fe/oxoglutarate-dependent dioxygenase of 4-coumaroyl CoA (C2′H) has been reported which is involved in the synthesis of umbelliferone, a central intermediate of coumarin derivatives. Among all the steps described for the synthesis of these molecules, the most critical is the prenylation of umbelliferone leading to the generation of demethylsuberosin (DMS) or osthenol. This enzymatic step is critical for two reasons. First, it is the entry point into the furanocoumarin pathway from umbelliferone, a common precursor in higher plants that produce furanocoumarins. This biosynthetic route is restricted to a small number of higher plants, suggesting limited distribution of prenyltransferase (PT). Second, the prenylation of umbelliferone at either C6 or C8 is a critical step, giving rise to either linear or angular furanocoumarins via the key prenylated intermediates DMS or osthenol, respectively.

Furanocoumarins are considered as natural toxins. Because of their properties, these chemicals have an important role in the protection against phytopathogens or insects. Furanocoumarins may also contribute to interspecific competition by inhibiting the germination and growth of neighboring plants. Moreover, when furanocoumarins are activated by UV light (especially UVA), they can lead to the acceleration of the tanning process and appearance of photodermatitis on human skin. In the mean time they are also involved in the production of free radicals which harm DNA, leading to an increased occurrence in the appearance of skin cancers. The use of essential oil containing furanocoumarins in perfume industry is therefore not recommended. The presence of these molecules can have a deleterious effect on skin, and can lead to the apparition of dermatitis.

Furanocoumarins are also involved in the inhibition of cytochrome P450s (CYP) that were described to metabolize endogenous and/or xenobiotic compounds in human liver. The well-known grapefruit juice-drug interaction is shown to be caused by some furanocoumarins which are inhibiting CYP3A4, a major P450 subtype responsible for drug metabolism in human. Geranyloxy derivatives of furanocoumarins responsible of the inhibition of CYP3A4 are notably bergamottin, epoxybergamottin, 6′,7′-dihydroxybergamottin (DHB), paradisin A (also called GF-I-1), paradisin B (also called GF-I-4), paradisin C (also called GF-I-6).

Due to their deleterious effects on human health by inhibiting enzymes, especially CYP3A4, or by increasing the risks of skin cancer, there exists a high demand for novel efficient method for inhibiting the production of furanocoumarins in plants.

The inventors have now discovered that inhibition of the expression of prenyltransferase protein in plants, in particular in grapefruit, leads to the inhibition of the synthesis of furanocoumarins. This discovery allows generating plants which are free of furanocoumarins, avoiding the appearance of deleterious effects on human health.

In Karamat et al. (The Plant Journal (2014), vol 77(4), pages 627-638), the authors have identified a membrane-bound prenyltransferase from parsley (PcPT), which has a strict substrate specificity towards umbelliferone and DMS, leading to linear furanocoumarins. The authors have demonstrated that PcPT is able to open the pathway to linear furanocoumarin but also to catalyze the synthesis of osthenol, the first intermediate of the angular furanocoumarin pathway. However, results obtained on parsley cannot be directly applied to grapefruit, due to the differences existing between these two species. Indeed, the authors of Karamat et al have unsuccessfully tried to modify the furanocoumarin pattern by over-expressing the PcPT protein in a Rutaceae plant, Ruta graveolens. For this, the authors have integrated the PcPT coding sequence of parsley into the genome of Ruta graveolens and monitored its expression using real-time quantitative PCR. The authors have noted that the production of furanocoumarin derivatives, in particular DMS, bergapten and osthenol was not significantly different from those obtained in wild-type R. graveolens. The authors have thus concluded that overexpressing the sequence encoding for the prenyltransferase of parsley (an apiaceae) doesn't impact the production of furanocoumarin in R. graveolens (a rutaceae). Using this sequence to modulate the production of furanocoumarins in citrus (a rutaceae) is therefore not a good solution. To regulate the production of these molecules in citrus species, it is necessary to identify a gene isolated from citrus plants which is involved in the synthesis of prenylated umbelliferone in these plants.

### FIGURES

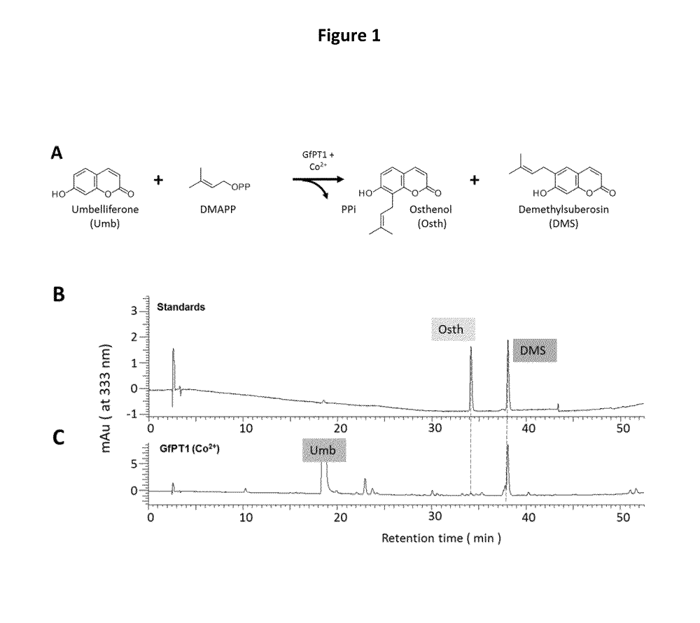

FIG. 1: Enzymatic characterization of GfPT expressed in Nicotiana benthamiana.
A) Expected reaction. Umbelliferone is transformed in osthenol and demethylsuberosin in presence of GfPT, DMAPP and Cobalt cations.
B) HPLC analysis of osthenol and DMS standard molecules.
C) HPLC analysis of the reaction mix of microsomes prepared from Nicotiana benthamiana leaves transiently over expressing GfPT (Karamat et al, Plant Journal Volume 77, Issue 4, Pages: 627-638) and incubated with Umbelliferone, DMAPP and Cobalt cations.
Umbelliferone is mainly transformed in DMS.

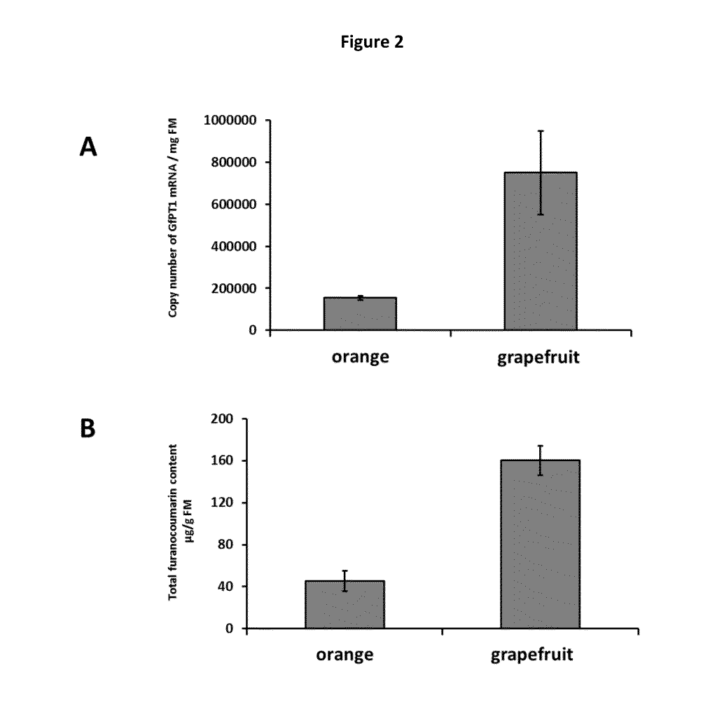

FIG. 2: Furanocoumarin contents and GfPT expression level in sweet orange and grapefruit.
A) Expression level of GfPT1 using real time quantitative RT-PCR
B) Quantitative analysis of the total furanocoumarin content in 2 days old fruits (21 different molecules were analyzed as described by Dugrand et al, Journal of Agriculture and Food Chemistry 61 (45), pp 10677-10684).

### DESCRIPTION OF THE INVENTION & DEFINITIONS

The present invention provides a novel and efficient method for inhibiting production of furanocoumarins in plants. Surprisingly, the inventors have discovered that plants, in particular plants belonging to Rutaceae family, with a defective GfPT protein are not able to produce furanocoumarins. In particular, the inventors have demonstrated that plants wherein the GfPT gene is inactivated or silenced produce less or no more furanocoumarins while plants expressing the GfPT gene produce furanocoumarins in large amount. More particularly, the inventors have demonstrated a total absence of furanocoumarin derivatives within plants wherein the GfPT gene is inactivated or silenced. As used therein, the term “furanocoumarin derivatives” designates all furanocoumarins compounds resulting from the umbelliferone prenylation, and in particular demethylsuberosin (DMS) or osthenol.

The present invention relates to a method for inhibiting the production of furanocoumarins in a plant, comprising the inhibition in said plant of the expression of a protein named GfPT, said GfPT protein having a coumarin-specific prenyltransferase activity and having at least 70% sequence identity with the polypeptide set forth in SEQ ID NO: 1.
As used therein, the term “GfPT protein” designates proteins containing a GfPT amino acid sequence and which have a coumarin-specific prenyltransferase activity. The abbreviation GfPT means GrapeFruit PrenylTransferase. Typically, GfPT protein plays an important role within the furanocoumarins biosynthetic pathway, since this protein allows the prenylation of umbelliferone into DMS in the presence of dimethylallyl-pyrophosphate (DMAPP). By inhibiting said GfPT protein, the authors have demonstrated that the biosynthetic pathway of furanocoumarins is inhibited, since the GfPT protein is no more able to catalyze the prenylation of umbelliferone into DMS. Preferably, the term “GfPT protein” designates grapefruit prenyltransferase 1 and is also called umbelliferone 6-dimethylallyltransferase. Preferred GfPT protein exhibits at least 70% sequence identity with the polypeptide set forth in SEQ ID NO: 1. The sequence identity and similarity values listed here are calculated by using the BLASTp program.
Within the context of the invention, the term “prenyltransferase function” indicates any activity mediated by a GfPT protein in a plant cell. The prenyltransferase function may be affected by the GfPT gene expression or the GfPT protein activity.
Within the context of the invention, the term “defective”, “inactivated”, “inactivation”, “inhibit” or “inhibition” in relation to the prenyltransferase function, indicate a reduction in the level of active GfPT protein in the cell or plant. Such reduction is typically of about 30%, more preferably 40%, as compared to the wild-type plant. Reduction may be more substantial (e.g. 50%, 60%, 70%, 80% or more) or complete (i.e. knock-out plants). More preferably, the reduction in the level of active GfPT protein is complete, leading to an absence of furanocoumarin derivatives within the plant.
In a specific embodiment of the invention, the GfPT protein has at least 75%, preferably at least 80% sequence identity with the polypeptide set forth in SEQ ID NO: 1. Preferably, the GfPT protein has at least 85%, at least 90% sequence identity with the polypeptide set forth in SEQ ID NO: 1. More preferably, the GfPT protein has at least 91%, at least 92%, at least 93%, at least 94%, at least 95%, at least 96%, at least 97%, at least 98%, at least 99% sequence identity with the polypeptide set forth in SEQ ID NO: 1. More preferably, the GfPT protein has the amino acid sequence of SEQ ID NO: 1.

According to the present invention, the expression of GfPT protein may be rendered defective or inhibited by various known techniques such as, for example, inactivation of the GfPT gene or of its promoter, or inactivation of a transcription factor regulating the expression of GfPT. Absence of expression of GfPT protein leads to an absence of production of furanocoumarin derivatives in plant. Indeed, absence of expression of GfPT protein leads to a negative regulation of the prenyltransferase function, leading to the inhibition of the umbelliferone prenylation step into DMS within the biosynthetic pathway of furanocoumarins.

Within the context of the invention, the term “GfPT gene” designates any nucleic acid that codes for a GfPT protein as defined above. The term “GfPT gene” includes GfPT DNA (e.g., genomic DNA) and GfPT RNA (e.g., mRNA). Specific example of a GfPT gene comprises the nucleic acid sequence of SEQ ID NO: 2.

Inactivation of prenyltransferase function may be carried out by techniques known per se in the art, such as without limitation, by genetic means, enzymatic techniques, chemicals methods, or combinations thereof. Inactivation may be conducted at the level of DNA, mRNA or protein, and inhibit the expression of GfPT gene or the activity of GfPT protein. Preferred inactivation methods affect the expression of GfPT gene and lead to the absence of production of GfPT protein in the cells. It should be noted that the inhibition of prenyltransferase function can be transient or permanent. Inhibition of the GfPT protein can be obtained by suppressing or decreasing its activity or by suppressing or decreasing the expression of the corresponding gene. Specifically, inhibition can be obtained via mutagenesis of the GfPT gene. For example, a mutation in the coding sequence can induce, depending upon the nature of the mutation, expression of an inactive protein, or of a reduced-active protein; a mutation at a splicing site can also alter or abolish the protein's function; a mutation in the promoter sequence can induce the absence of expression of said protein, or the decrease of its expression. Mutagenesis can be performed, e.g., by suppressing all or part of the coding sequence or of the GfPT promoter, or by inserting an exogenous sequence, e.g., a transposon, into said coding sequence or said promoter. It can also be performed by inducing point mutations, e.g., using ethyl methanesulfonate (EMS) mutagenesis or radiation. The mutated alleles can be detected, e.g., by PCR, by using specific primers of the GfPT gene.
Various high-throughput mutagenesis and splicing methods are described in the prior art. By way of examples, we may cite “TILLING” (Targeting Induced Local Lesions In Genome)-type methods, described by Till, Comai and Henikoff (2007) (R. K. Varshney and R. Tuberosa (eds.), Genomics-Assisted Crop Improvement: Vol. 1: Genomics Approaches and Platforms, 333-349.).

Plants comprising a mutation in the GfPT gene that induces inhibition of the GfPT protein are also part of the goal of the present invention. This mutation can be, e.g., a deletion of all or part of the coding sequence or of the GfPT promoter, or it may be a point mutation of said coding sequence or of said promoter.
Advantageously, inhibition of the GfPT protein is obtained by silencing or by knock-out techniques on

GfPT gene. Various techniques for silencing genes in plants are known. Antisense inhibition or co suppression, described, e.g., in Hamilton and Baulcombe, 1999, Science, vol 286, pp 950-952, is noteworthy. It is also possible to use ribozymes targeting the mRNA of the GfPT protein. Preferably, silencing of the GfPT gene is induced by RNA interference targeting said gene. An interfering RNA (iRNA) is a small RNA that can silence a target gene in a sequence-specific way. Interfering RNA include, specifically, “small interfering RNA” (siRNA) and micro-RNA (miRNA). The most widely-used constructions lead to the synthesis of a pre-miRNA in which the target sequence is present in sens and antisens orientation and separated by a short spacing region. The sens and antisens sequence can hybridize together leading to the formation of a hairpin structure called the pre miRNA. This hairpin structure is maturated leading to the production of the final miRNA. This miRNA will hybridize to the target mRNA which will be cleaved or degraded, as described in Schwab et al (Schwab et al, 2006 The Plant Cell, Vol. 18, 1121-1133) or in Ossowski et al (Ossowski et al, 2008, The plant Journal 53, 674-690).

Inhibition of the GfPT protein can also be obtained by gene editing of GfPT gene. Various methods can be used for gene editing, by using transcription activator-like effector nucleases (TALENs), clustered Regularly Interspaced Short Palindromic Repeats (CRISPR/Cas9) or zinc-finger nucleases (ZFN) techniques (as described in Belhaj et al, 2013, Plant Methods, vol 9, p 39, Chen et al, 2014 Methods Volume 69, Issue 1, p 2-8). Preferably, the inhibition of the GfPT protein is obtained by using clustered Regularly Interspaced Short Palindromic Repeats (CRISPR/Cas9). The use of this technology in genome editing is well described in the art, for example in Fauser et al. (Fauser et al, 2014, The Plant Journal, Vol 79, p 348-359), and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Cas9 is thus the hallmark protein of the type II CRISPR-Cas system, and a large monomeric DNA nuclease guided to a DNA target sequence adjacent to the PAM (protospacer adjacent motif) sequence motif by a complex of two noncoding RNAs: CRIPSR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used. The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Cas9 expression plasmids for use in the methods of the invention can be constructed as described in the art.

The absence of prenyltransferase function in modified engineered plants or plant cells can be verified based on the phenotypic characteristics of their offspring; homozygous plants or plant cells for a mutation inactivating the GfPT gene have a content of furanocoumarin rate that is lower than that of the wild plants (not carrying the mutation in the GfPTgene) from which they originated. Generally, this furanocoumarin rate is at least 10 times lower, preferably at least 20 times lower, at least preferably 30 times lower, preferably at least 40 times lower, preferably at least 50 times lower than that of the wild plants from which they originated. More preferably, this furanocoumarin rate is at least 60 times lower, at least 70 times lower, at least 80 times lower, at least 90 times lower than that of the wild plants from which they originated. More preferably, this furanocoumarin rate is at least 100 times lower than that of the wild plants from which they originated. Preferably, the furanocoumarin rate is null or equal to zero compared to the wild plants from which they originated.

The present invention also relates to a DNA construct capable of inhibiting the expression of a GfPT protein of which the polypeptide sequence has at least 70% identity with the sequence SEQ ID No. 1. Preferably, the DNA construct comprises one or more polynucleotides capable of inhibiting the expression of a GfPT protein of which the polypeptide sequence has at least 80%, preferably at least 85%, preferably 90% identity with the sequence SEQ ID No. 1. More preferably, the DNA construct comprises one or more polynucleotides capable of inhibiting the expression of a GfPT protein of which the polypeptide sequence has at least 91%, at least 92%, at least 93%, at least 94%, at least 95%, at least 96%, at least 97%, at least 98%, at least 99% sequence identity with the polypeptide set forth in SEQ ID NO: 1. More preferably, the GfPT protein has the amino acid sequence of SEQ ID NO: 1. The sequence identity and similarity values listed here are calculated by using the BLASTp program.
In a specific embodiment of the invention, the polynucleotide of the DNA construct encodes an antisense RNA, an interfering RNA, a micro-RNA, a ribozyme targeting the GfPT gene, a complex

RNA-guided Cas9 nuclease targeting the GfPT gene or a nuclease targeting the GfPT gene. The present invention also relates to an expression cassette said cassette including one or more DNA constructs, whose transcript of the DNA construct is a complex RNA-guided Cas9 nuclease targeting the GfPT gene, placed under the transcriptional control of functional promoter in a plant cell.

A broad selection of appropriate promoters for expressing heterologous genes in plant cells or in plants useful according to the invention is available in the prior art.
These promoters can be obtained, e.g., from plants, plant viruses, or bacteria such as Agrobacterium. They include constitutive promoters namely, promoters that are active in most tissues and cells and under most environmental conditions as well as tissue-specific or cell-specific promoters, which are only active or primarily active in certain tissues or certain types of cells, and inducible promoters that are activated by physical or chemical stimuli. Examples of constitutive promoters that are currently used in plant cells are the 35S promoter of the cauliflower mosaic virus (CaMV), or derivatives thereof, the cassava vein mosaic virus (CsVMV), the maize ubiquitin promoter, or the rice “Actin-Intron-actin” promoter.
The expression cassettes of the invention generally include a transcriptional terminator, such as the nopalin synthase (NOS) terminator or the Arabidopsis Heat Shock Protein (HSP) terminator. They may also include other transcription-regulating elements such as amplifiers.

The DNA construct of the invention also encompass recombinant vectors comprising an expression cassette of the invention. These recombinant vectors may also include one or several marker genes, which enable the selection of the transformed cells or plants. The selection of the most appropriate vector depends, in particular, on the expected host and on the anticipated method to be used for transforming the relevant host. Numerous methods for genetic transformation of plant cells or plants are available in the prior art, for numerous dicotyledonous or monocotyledonous plant species. By way of non-limiting examples, we may mention virus-mediated transformation, transformation by microinjection or by electroporation, transformation by microprojectiles, transformation by Agrobacterium, etc.

The present invention also relates to host cell comprising the recombinant vector of the invention. Said host cell can be a prokaryote cell, e.g., an Agrobacterium cell, or a eukaryote cell, e.g., a plant cell that has been genetically transformed by a DNA construction of the invention. The construction can be expressed transiently; it can also be incorporated into a stable extrachromosomal replicon, or integrated into the chromosome.

The present invention also relates to a plant or plant cells, which have been engineered to produce less furanocoumarins derivatives than wild-type plant. Preferably, the plant or plant cells of the invention fails to produce furanocoumarins derivatives. The content of furanocoumarin present in said plant or plant cells is null or equal to zero. The plant or plant cells of the invention exhibit a GfPT gene which is defective or inactivated. The plant or cell plants of the invention exhibiting a defective GfPT gene result of an engineered alteration of the plant genome. By way of non-limiting examples of alteration of plant genome resulting in defective GfPT gene, we may mention deletion, insertion and/or substitution of one or more nucleotides, site-specific mutagenesis, ethyl methanesulfonate (EMS) mutagenesis, targeting induced local lesions in genomes (TILLING), knock-out techniques, gene editing techniques, for example by using CRISPR/Cas9, TALEN or ZFN techniques, or by gene silencing induced by RNA interference. In a preferred embodiment, plant or plant cells of the invention are obtained by using gene editing techniques, such as CRISPR/Cas9, TALEN or ZFN techniques, and in particular by using CRISPR/Cas9 technique.
Within the context of the invention, the term “plant”, “plantlet” or “plant cells” designates plants, plantlet or plant cells belonging to Rutaceae families. Preferably, plants belong to the Citrus group. By way of non-limiting examples, we may mention grapefruit, pummelo, bergamot, papeda, lime. More preferably, plants of the invention are grapefruit.

The invention also related to seeds of plants of the invention, as well as to plants, or descendants of plants grown or otherwise derived from said seeds, said plants having a content of furanocoumarins which is equal to zero or null.

The present invention additionally provides a method for inhibiting expression of furanocoumarins in a plant, said method comprising:
inactivating GfPT gene encoding for the GfPT protein in plant cells;
cultivating said plant cells and regenerating the resulting plantlet;
selecting the plantlet exhibiting inactivated GfPT gene; and
growing said plant, whereby expression of the GfPT protein is inhibited.

Selection of plantlet or plant exhibiting an inactivated GfPT gene can be made by techniques known per se to the skilled person (e.g., PCR, hybridization, use of selectable marker gene, protein dosing, western blot, HPLC, UPLC etc.). Plant generation from the modified plant cells can obtain by using methods known per se to the skilled worker. In particular, it is possible to induce, from callus cultures or other undifferentiated cell biomasses, the formation of shoots and roots. The plantlets thus obtained can be planted out and used for cultivation. Methods for regenerating plants from cells are described, for example, by Mendez da Gloria et al (Mendez da Gloria et al, (2000), Pesq. agropec. bras. vol.35 no.4, p727-732) or Singh et al (Singh et al, (2011) Physiol Mol Biol Plants. 2vol 17(2), p 161-169). The resulting plants can be bred and hybridized according to techniques known in the art. Preferably, two or more generations should be grown in order to ensure that the genotype or phenotype is stable and hereditary.

The inactivation of GfPT gene in said plant can be performed by deletion, insertion and/or substitution of one or more nucleotides, site-specific mutagenesis, ethyl methanesulfonate (EMS) mutagenesis, targeting induced local lesions in genomes (TILLING), knock-out techniques, gene editing techniques, for example by using CRISPR/Cas9, TALEN or ZFN techniques, or by gene silencing induced by RNA interference. In a preferred embodiment, inactivation of GfPT gene of the invention is obtained by using gene editing techniques, such as CRISPR/Cas9, TALEN or ZFN techniques, and in particular by using CRISPR/Cas9 technique.

The present invention additionally provides a method for inhibiting expression of furanocoumarins in a plant, said method comprising the following steps:
transforming a plant cell by integrating into a plant genome a recombinant vector comprising an expression cassette, wherein the expression cassette comprises a DNA construct comprising one or more polynucleotides capable of inhibiting the expression of a GfPT protein of which the polypeptide sequence has at least 70% identity with the sequence SEQ ID No. 1,
cultivating said transformed plant cells in order to regenerate a plantlet;
selecting plantlet that has in its genome said expression cassette; and
growing said plant, whereby expression of the GfPT protein is inhibited. The expression of the DNA construct of the invention leads to the total inhibition of expression of the GfPT protein, which confers to said plant the inability to produce furanocoumarin derivatives.

The present invention additionally relates to an isolated DNA molecule, encoding coumarin-specific prenyltransferase, wherein the DNA molecule has at least 40% sequence similarity to SEQ ID NO: 2 and wherein the DNA molecule encodes an amino acid sequence that has coumarin-specific prenyltransferase activity and has at least 70% sequence similarity to SEQ ID NO:1. Preferably, the DNA molecule has at least 45%, preferably 50%, preferably 55%, preferably 60% sequence similarity to SEQ ID NO: 2. More preferably, the DNA molecule has at least 65%, preferably 70%, preferably 75%, preferably 80% sequence similarity to SEQ ID NO: 2. In a specific embodiment of the invention, the DNA molecule has at least 85%, preferably 90%, preferably 95%, preferably 96%, preferably 97%, preferably 98%, preferably 99% sequence similarity to SEQ ID NO: 2. The sequence identity and similarity values listed here are calculated by using the BLASTn program.
In another specific embodiment of the invention, the DNA molecule has the sequence SEQ ID NO: 2.

### EXAMPLES

### Example 1

### Identification of the Prenyltransferase Function of GfPT Protein in Grapefruit

1. Construction of Binary Vector and Agrobacterium tumefaciens Strains

The ORF of GfPT was first cloned into the pMD 19 plasmid (Clontech) according to the suppliers recommendations and further subcloned into the pRI201 plasmid (Takara) using Barn HI and Sal I restriction enzyme to create the pRI201-GfPT plasmid. The recombinant pRI201-GfPT plasmid was introduced into A. tumefaciens strain LBA4404. Agrobacterium strain C5851 containing pBIN61-P19 (Voinnet et al., 2003), provided by D. Baulcombe (Department of Plant Science, University of Cambridge, UK), and the transformed A. tumefaciens strain LBA4404 were used for transient expression in N. benthamiana plants.

2. Heterologous Expression of GfPT in N. benthamiana

Nicotiana benthamiana plants were used for infiltration experiments as described by Karamat et al., 2014. Bacterial strains were individually plated on YEB medium (5.0 g L⁻¹ sucrose, 5.0 g L⁻¹ peptone, 5.0 g L⁻¹ beef extract, 1.0 g L⁻¹ yeast extract, 0.049 g L⁻¹ MgSO4 7 H 2 O, 10 g L⁻¹ agar, pH adjusted to 7.2) in the presence of antibiotics (100 mg L⁻¹ rifampicine and 50 mg L⁻¹ kanamycin), and incubated at 30° C. for 2 days. Colonies were inoculated into 10 ml liquid YEB medium in the presence of antibiotics and incubated at 30° C. for 18 h. The resulting bacterial culture was pelleted by 5 min centrifugation at 5000 g, followed by three successive washes with sterilized distilled water by re-suspending the pellet in sterile water and centrifuged at 5000 g for 5 min. The pellet was finally re-suspended in water to adjust the OD₆₀₀ to between 0.3 and 0.4. Agrobacterium (strain LBA4404) containing pRI201-GfPT were infiltrated together with the C5851 strain containing pBIN61-P19, into N. benthamiana leaves. Leaves inoculated with pRI201-GfPT were used for microsomal preparation.

3. Microsomal Preparation from N. benthamiana Leaves

Inoculated leaves of N. benthamiana were used for extraction of membranous proteins. The leaves were homogenized using a PolyTron PT2100 (Kinematia A G, www kinematica.ch) in 0.1 M potassium phosphate buffer (pH 7.0) containing 10 mM dithiothreitol and a cocktail of protease inhibitors (Complete Mini, EDTA-free; Roche, www.roche-applied-science.com). Polyvinylpolypyrrolidone (0.1 g per 1 g of leaves) was added to the homogenate. Samples were then centrifuged at 10 000 g for 30 min to separate the supernatants and pellets, and were filtered through Miracloth (Merck-Millipore, www.merckmillipore.com). Recombinant proteins present in supernatants were collected by ultracentrifugation performed for 1 h at 100 000 g. The pellet of crude membranes was resuspended in 500 μl of 50 mM Tris-HCl (pH 8.0).

4. In vitro Enzymatic Assay and HPLC Analysis

The enzyme assay reactions contained 1 μg of total protein, 500 μM Tris-HCl, pH 8.0, 1 mM Co²⁺, and substrates at concentrations ranging from 0.2 to 1 mM for DMAPP and from 2 to 500 μM for umbelliferone. All assays were performed in triplicate. The reactions were incubated at 25° C. for 2 h, and were stopped by addition of 1 μl trifluoroacetic acid. The reaction mixtures were analyzed after centrifugation at 16 000 g for 10 min by HPLC-Diode Array Detector on a Cosmosil 5C18-AR-II column (Nacalai Tesque Inc., www.nacalai.co.jp) using a linear gradient of 10-70% methanol for 35 min at 1 ml min⁻¹. The UV spectra and retention times of the products were compared to standard moelcules.
Umbelliferone was detected at 333 nm, DMS was detected at 333 nm, and osthenol was detected at 333 nm (See FIG. 1). The kinetic parameters were calculated using the SigmaPlot software program (Systat Software Inc., www.sigmaplot.com/)

The results described in FIG. 1 clearly show a metabolization of umbelliferone leading to the synthesis of DMS and Osthenol.

5. Real-Time PCR

The expression level of the GfPT gene in grapefruit and sweet orange was assessed by Real Time Quantitative RT-PCR (See FIG. 2).
We could establish a relationship between the expression level of the GfPT gene and the furanocoumarin content in sweet orange and grapefruit (See FIG. 2).

1. A method for inhibiting production of furanocoumarins in a plant, comprising the inhibition in said plant of the expression of a protein named GfPT, said GfPT protein having coumarin-specific prenyltransferase activity and having at least 70% sequence identity with the polypeptide set forth in SEQ ID NO: 1.

2. The method of claim 1 wherein the GfPT protein has at least 80% sequence identity with the polypeptide set forth in SEQ ID NO: 1.

3. The method of claim 1 wherein the GfPT protein has at least 90% sequence identity with the polypeptide set forth in SEQ ID NO: 1.

4. The method according to claim 1, wherein the GfPT protein has the amino acid sequence of SEQ ID NO: 1.

5. The method according to claim 1, wherein the inhibition of the expression of GfPT protein is obtained by inactivation of the GfPT gene or of its promoter, or a transcription factor regulating the expression of GfPT.

6. The method according to claim 5, wherein said GfPT gene is inactivated by a deletion, insertion and/or substitution of one or more nucleotides, site-specific mutagenesis, ethyl methanesulfonate (EMS) mutagenesis, targeting induced local lesions in genomes (TILLING), knock-out techniques, gene editing techniques, for example by using CRISPR/Cas9, TALEN or ZFN techniques, or by gene silencing induced by RNA interference.

7. The method according to claim 1, wherein said plant belongs to the Rutaceae family, in particular to the Citrus group.

8. The method according to claim 7, wherein the plant is selected among the group of a grapefruit, pummelo, bergamot, papeda or lime.

9. A DNA construct, comprising one or more polynucleotides capable of inhibiting the expression of a GfPT protein of which the polypeptide sequence has at least 70% identity with the sequence SEQ ID No. 1.

10. The DNA construct as claimed in claim 9, wherein said polynucleotide encodes an antisense RNA, an interfering RNA, a micro-RNA, a ribozyme targeting the GfPT gene, a complex RNA-guided Cas9 nuclease targeting the GfPT gene, TALEN nuclease targeting the GfPT gene or a ZFN nuclease targeting the GfPT gene.

11. An expression cassette, comprising one or more DNA constructs as defined in claim 9 placed under the transcriptional control of functional promoter in a plant cell.

12. An expression cassette according to claim 11, wherein the transcript of the DNA construct is a complex RNA-guided Cas9 nuclease targeting the GfPT gene.

13. A recombinant vector comprising an expression cassette according to claim 11.

14. A host cell comprising a recombinant vector according to claim 13.

15. A plant, wherein said GfPT gene is defective as a result of a deletion, insertion and/or substitution of one or more nucleotides, site-specific mutagenesis, ethyl methanesulfonate (EMS) mutagenesis, targeting induced local lesions in genomes (TILLING), knock-out techniques, gene editing techniques, for example by using CRISPR/Cas9, TALEN or ZFN techniques, or by gene silencing induced by RNA interference.

16. The plant according to claim 15, wherein said plant belongs to the Rutaceae family, in particular to the Citrus group.

17. The plant according to claim 16, wherein the plant is selected among the group of a grapefruit, pummelo, bergamot, papeda or lime.

18. A method for inhibiting expression of furanocoumarins in a plant, said method comprising the following steps:
inactivating GfPT gene encoding for the GfPT protein in plant cells;
cultivating said plant cells and regenerating the plantlet;
selecting the plantlet exhibiting inactivated GfPT gene; and
growing said plant,
whereby expression of the GfPT protein is inhibited.

19. A method for inhibiting expression of furanocoumarins in a plant according to claim 18, wherein GfPT gene is inactivated by deletion, insertion and/or substitution of one or more nucleotides, site-specific mutagenesis, ethyl methanesulfonate (EMS) mutagenesis, targeting induced local lesions in genomes (TILLING), knock-out techniques, gene editing techniques, for example by using CRISPR/Cas9, TALEN or ZFN techniques, or by gene silencing induced by RNA interference.

20. The method according to claim 18, wherein said plant belongs to the Rutaceae family, in particular to the Citrus group.

21. The method according to claim 20, wherein the plant is selected among the group of a grapefruit, pummelo, bergamot, papeda or lime.

22. A method for inhibiting expression of furanocoumarins in a plant, said method comprising the steps of:
transforming a plant cell by integrating into a plant genome a recombinant vector comprising an expression cassette, wherein the expression cassette comprises a DNA construct comprising one or more polynucleotides capable of inhibiting the expression of a GfPT protein of which the polypeptide sequence has at least 70% identity with the sequence SEQ ID No. 1,
cultivating said transformed plant cells in order to regenerate a plantlet;
selecting plantlet that has in its genome said expression cassette; and
growing said plant, whereby expression of the GfPT protein is inhibited.

23. An isolated DNA molecule, encoding coumarin-specific prenyltransferase, wherein the DNA molecule has at least 40% sequence similarity to SEQ ID NO: 2 and wherein the DNA molecule encodes an amino acid sequence that has coumarin-specific prenyltransferase activity and has at least 70% sequence similarity to SEQ ID NO:1.

24. An isolated DNA molecule, encoding coumarin-specific prenyltransferase, wherein the DNA molecule has the sequence SEQ ID NO: 2 and wherein the DNA molecule encodes an amino acid sequence that has coumarin-specific prenyltransferase activity and has at least 70% sequence similarity to SEQ ID NO:1.


SEQUENCE LISTING (2 sequences)

SEQ ID NO: 1 - protein, 397 aa, Grapefruit
MSPLMHLHSGFSPKYHPNLQRSGCNKTLESPLVQGRRKSVKCSRSPFYLTSKIRKSEDISNESCKLMFNRPVTLQVCYASKSEDANQRMTSQEVFFKNLDALHRFIRPYTLMGTVIAITSVSLLPLQNLDDLTPTYFMGFFKAMVPGLLMTVYEVAINQLYDVKIDKVNKPNLPLTSGDLSMRTGIAIASSSLLMSLAMGIMLRSPPFLLALIIWFLLASAYSADLPFLRWKRSSFLTTLYIVLERGLLLQFAYFIHIQKYVLGRPIAITRTLMFAVAITCCFCFVISVLKDIPDEDGDREFGIRTLSVILGKESVLWLCVYVLFIAYGAAVIVGLTSSPYLLSKLVMIISHSMLATLLWHQARTVDLSSKASTLSFYMFIWKLHYVECLIIPFVRL

SEQ ID NO: 2 - DNA, 1194 bp, Grapefruit
atgagtcctctcatgcatttacattcaggcttctctccaaagtatcatcctaatttgcaacggtctggttgcaataaaacgcttgaatcgccactagttcaaggcaggagaaaatctgtgaaatgctcccgaagtccattttatttgaccagtaaaattagaaagagtgaggatataagcaatgaaagctgtaaattaatgttcaaccgtccggtcaccttacaagtttgttatgcatcaaaatccgaggatgctaatcaaaggatgacctctcaggaagttttcttcaagaatttagatgcactccaccgttttattcgtccctacacacttatgggcactgtaattgctataacatcggtttctcttcttcccctacaaaatcttgatgatttgactcccacatatttcatgggattttttaaggccatggtgcccggattgctgatgaccgtttatgaggttgctataaaccagttgtatgatgttaaaatagacaaggtcaacaagcctaatctcccccttacttctggtgacctgtccatgcgaactggaatagcaattgcttcctcatccttattgatgagtctggccatgggaattatgcttcgatctccaccgtttcttttggccctcatcatatggtttctccttgcaagtgcttattctgcggatcttccctttcttaggtggaagagaagttcatttctaactacattgtacatcgtgctagagaggggacttctactccaatttgcttacttcatacacattcagaaatacgtacttggaagaccaatagcaattacgagaacattgatgtttgcagtcgctattacgtgttgtttctgctttgtgatttcagttctcaaggatatacctgatgaggatggggatagagaatttggcattcgaacgcttagtgtcattttagggaaagaaagtgtactttggctttgtgtttatgtgctgttcattgcttatggagccgctgttatagtcggattaacttcttcaccttacctgctgagcaaacttgtcatgataattagccacagcatgctagctactcttctgtggcatcaggctcgaactgttgatctctctagtaaagcgtcaacactttctttttacatgttcatttggaagttacactatgttgaatgcctcattattccatttgtacgattatga